(19) United States
(12) Patent Application Publication
(10) Pub. No.: US 2007/0280959 A1
(43) Pub. Date: Dec. 6, 2007

(54) Use Of Platelets Or Platelet Rich Plasma(Prp)

(76) Inventors: Thomas Meury, St. Hyacinthe (CA); Thierry Stoll, Meinisberg (CH); Mauro Alini, Davos Platz (CH)

Correspondence Address: JONES DAY, 222 EAST 41ST STREET, NEW YORK, NY, 10017-6702, US

(21) Appl. No.: 11/568,674

(22) PCT Filed: May 5, 2004
(86) PCT No.: PCT/CH04/00271

§ 371 Date: Apr. 23, 2007

(51) Int. Cl. A61K 35/14; A61K 35/16; A61K 38/18; A61P 17/00; A61P 19/08; C12N 5/06

(52) U.S. Cl. 424/198.1; 424/530; 424/532; 435/325

(57) ABSTRACT

A new use is disclosed for the content of platelets or platelet rich plasma (PRP) obtained by disruption of their membranes for the preparation of an agent for the treatment of bone, cartilage or skin.

The invention relates to the use of platelets or platelet rich plasma (PRP) according to the preamble of claim 1 and to a method for preparing an agent for the treatment of bone, cartilage or skin according to the preamble of claim 9.

Platelets are known to release several mediators and cytokines upon activation but not for treatment of bone, cartilage or skin.

The invention is based on the objective of providing new uses and a much easier and quicker method for activation of PRP with comparable results to prior art methods.

The invention solves the posed problem by the characterizing features of independent claims 1 and 9. The advantages achieved by the method according to the invention are essentially to be seen in the fact that it is much less time consuming since it required only about 1 minute, compared to approximately 15-30 minutes according to prior art methods. A further advantage lies in the fact that no non-autologous component is involved in the method.

The treatment may be aimed at curing diseases or defects in bone, cartilage or skin as well as at the filling of defects in or the replacement of bone, cartilage or skin. The treatment may also activate, accelerate or stimulate growth of bone, cartilage or skin as well as

In a special embodiment said agent comprises one or more of the following substances: platelet derived growth factor AB (PDGF-AB), platelet derived growth factor M (PDGF-M), platelet derived growth factor BB (PDGF-BB), vascular endothelial growth factor (VEGF), transforming growth factor β (TGF-β), epidermal growth factor (EGF), insulin-like growth factor (IGF), epithelial cells growth factor ECGF and fibroblastic growth factor (FGF). These substances improve the wound healing. Preferably said agent does not comprise additional organic compounds and in particular not any ionophores in order to keep toxicity low.

Preferably said agent should neither contain any compounds from another species or from another individual than said platelets or platelet rich plasma (PRP). The acceptance by the patient is enhanced by this measure.

In a special embodiment of the method for preparing an agent for the treatment of bone, cartilage or skin diseases or defects sonification is performed at a temperature between 0° C. and +10° C., preferably between 0° C. and +2° C. Preferably the sonification is performed on ice.

Sonification may be performed at a frequency between 10 to 30 kHz, preferably between 15 and 25 kHz.

The duration of the sonification may range from 1 to 50 second, preferably from 5 to 20 seconds.

Preferably no other platelet disrupting treatment is performed either before or after the sonification.

The ex vivo activation of platelets or platelet rich plasma (PRP) can be done chemically or physically including addition of bovine thrombin, repeated freeze-thaw cycles, or the addition of a ionophore. The use of chemicals like bovine thrombin or ionophore is questionable and the repeated freeze-thaw cycles are time consuming. The preferred method for obtaining an agent for the treatment of bone, cartilage or skin disease according to the invention is sonification. Sonification has proved to be a simple yet quick and powerful method to achieve PRP activation. Activation efficiency is comparable to the known methods, but sonification is less time-consuming than freeze-thaw cycles and does not use potentially dangerous chemicals like bovine thrombin.

The invention and additional modifications of the invention are explained in even more detail with reference to a number of specific examples.

### EXAMPLE 1

### Preparation

Citrate-anticoagulated was spinned down at 200 g for 10 min at room temperature. The upper layer was transferred to a new tube and was centrifuged again at 200 g for 20 min at room temperature. The top 30-50% of the supernatant (platelet poor plasma, PPP) were removed and the pellet was resuspended in the remaining supernatant, producing platelet rich plasma (PRP). The PRP was combined with an equal volume of CaCl₂ (10% final conc.). Sonication of PRP was performed on ice by applying a frequency of 25 kHz for 15 sec. This caused the platelets to release the mediators and cytokines. It took 1 hour for the gel to solidify. All steps were performed under sterile conditions.

### EXAMPLE 2

### Preparation

Anticoagulated blood was spinned down at 150-400 g, preferably 200 g for 10 to 60 min at room temperature (slow brake settings). The upper layer (plasma) was transferred to a new tube and centrifuged again at 500-2000 g for 7-10 min at room temperature. The top 50-70 vol-% of the supernatant (containing PPP, platelet-poor-plasma) was removed and platelet-rich plasma (PRP) was produced by resuspending the pellet in the remaining supernatant by vortexing. This method produced 7-15 mL non-activated PRP out of 80-130 mL of unprocessed blood. This PRP was stored at 2-8° C. for up to 24 hours, preferably less than 8 hours. 10% (final vol.) of CaCl₂ were added to the PRP, which inhibited the anticoagulation effect of the citrate and allowed polymerization of fibrinogen into a fibrin gel. Sonification of the PRP was performed on ice by applying a frequency of 20 kHz for 2×5 sec. This caused the platelets to degranulate and release the mediators and cytokines. It took 1-2 hours for the gel to solidify. All these steps were performed under sterile conditions.

### EXAMPLE 3

PRP was prepared in the way described above in Examples 1 or 2 from blood collected in the immediate preoperative period. It was stored at 2-8° C. and used within 1 hour of preparation for technical reasons (before the PRP gels). RP prepared in the way described in Examples 1 or 2 was applied directly to a bone or cartilage defect using a syringe. The amount of PRP applied was dependent of the size of the bone or cartilage defect, but generally as much PRP as possible was used (100 mL blood resulted in about 10 mL PRP). To improve results, the PRP was further concentrated by removing more supernatant after the second centrifugation step (200-2000 g, as described in Example 2) and resuspending the platelet pellet in less volume, before activation by sonification.

### EXAMPLE 4

PRP alone (as prepared according to Example 2) or combined with a bone marrow aspirate was added to a bone implant as an initial source of growth factors regulating proliferation, differentiation, chemotaxis, and morphogenesis of cells and tissue. This resulted in less bleeding and a significantly increased osteoconduction and therefore in faster and better healing of the bone defect treated in this way. Up to 100% more bone tissue in PRP treated implants compared to untreated implants 6 months after implantation was obtained.

### EXAMPLE 5

### Protein Levels

Activation of PRP by sonication produced VEGF, PDGF-AB, PDGF-BB and TGF-β levels, that are comparable (±30%) to the respective levels produced by the other methods (Thrombin, Freeze/Thaw)

### EXAMPLE 6

### In Vitro

In vitro experiments using human bone marrow stromal cells (hBMSC) cultured in IMDM, 10% FBS, 10 mM β-Glycerophosphate, 0.1 mM Ascorbic Acid-2-Phosphate and 10% PRP showed significantly higher matrix mineralization (measured by ⁴⁵Ca²⁺ incorporation) after 14 days of culture than hBMSC grown without PRP. The addition of PRP produced by sonication resulted in up to 20× more Ca incorporation, the freeze/thaw method showed similar results, while Thrombin-activated PRP only resulted in a 10× increase, compared to hBMSC grown without PRP.

In vitro experiments also showed (same setup as above), that the addition of PRP to hBMSC results in a 75-100% increase in hBMSC proliferation (estimated by measuring the DNA content), independent of the PRP activation method.

1. A method for the treatment of a bone, cartilage or skin disease or defect, comprising administering to a subject an agent comprising the content of platelets or platelet rich plasma (PRP) obtained by disruption of the membranes of said platelets.

2. (canceled)

3. The method of claim 1, which is aimed at activating, accelerating or stimulating growth of bone, cartilage or skin.

4. The method of claim 1, which is aimed at H filling of defects in or replacing bone, cartilage or skin.

5. The method of claim 1, wherein said agent comprises one or more substance selected from the group consisting of platelet derived growth factor AB (PDGF-AB), platelet derived growth factor AA (PDGF-AA), platelet derived growth factor BB (PDGF-BB), vascular endothelial growth factor (VEGF), transforming growth factor β (TGF-β), epidermal growth factor (EGF), insulin-like growth factor (IGF), epithelial cells growth factor (ECGF), and fibroblastic growth factor (FGF).

6. The method of claim 1, wherein said agent does not comprise additional organic compounds.

7. The method of claim 1, wherein said agent does not comprise any ionophores.

8. The method of claim 1, wherein said agent does not contain any compounds from another species or from another subject.

9. A method for preparing an agent for the treatment of a bone, cartilage or skin disease or defect, comprising sonicating platelets or platelet rich plasma (PRP) at a temperature between 0° C. and +37° C. to disrupt the platelet membranes and a release the content of said platelet.

10. The method of claim 9, wherein said sonicating is performed at a temperature between 0° C. and +10° C., or between 0° C. and +2° C.

11. The method of claim 9, wherein said sonicating is performed on ice.

12. The method of claim 9, wherein said sonicating is performed at a frequency between 10 to 30 kHz, or between 15 and 25 kHz.

13. The method of claim 9, wherein said sonicating is performed during 1 to 50 second, preferably or during 5 to 20 seconds.

14. The method of claim 9, wherein no other platelet disrupting treatment is performed either before or after said sonicating.
